(19) United States
(12) Patent Application Publication
(10) Pub. No.: US 2025/0262226 A1
(43) Pub. Date: Aug. 21, 2025

(54) COMBINATIONS OF VITAMIN D3, NIACINAMIDE AND LIPOIC ACID FOR USE IN THE MAINTENANCE OF HEALTHY BLOOD GLUCOSE LEVELS

(71) Applicants: Satishchandran Chandrasekhar, Jupiter, FL (US); Uday Saxena, Telengana (IN)

(72) Inventors: Satishchandran Chandrasekhar, Jupiter, FL (US); Uday Saxena, Telengana (IN)

(21) Appl. No.: 18/728,545

(22) PCT Filed: Jan. 13, 2023
(86) PCT No.: PCT/US2023/010800

§ 371 Date: Jul. 12, 2024

(30) Foreign Application Priority Data
Jan. 17, 2022 (IN) 202241002534

(51) Int. Cl. A61K 31/593; A61K 9/48; A61K 31/385; A61K 31/455; A61P 3/10

(52) U.S. Cl. CPC A61K 31/593; A61K 9/485; A61K 31/385; A61K 31/455; A61P 3/10

(57) ABSTRACT

Provided is a health supplement product that can be administered to subjects with pre-diabetes and type 2 diabetes, the product comprising a fixed dose combination of vitamin D3, niacinamide (which is a form of vitamin B3), and lipoic acid (which is an antioxidant) that improves glucose uptake by muscle cells. The compositions address critical molecular steps in muscle glucose uptake and metabolism to result in blood glucose and A1C lowering. In vitro and in vivo data support the use of the combination for glucose control in subjects having prediabetes and as a supplement to other therapeutic treatments in type 2 diabetics.

### RELATED APPLICATION

Benefit of priority is claimed to Indian Provisional Application 20/224,1002534, filed with the Indian Patent Office (The Office of the Controller General of Patents, Designs and Trade Marks) on Jan. 17, 2022, and titled “Novel Triple Action Fixed Dose Combination to Maintain Healthy Blood Glucose Levels,” and where permitted, the subject matter of which is incorporated by reference in its entirety.

### FIELD OF INVENTION

The present invention relates to a health supplement product for treating or preventing the complications of pre-diabetes and type 2 diabetes, comprising a fixed dose combination of Vitamin D3, Niacinamide, a form of Vitamin B3, and lipoic acid, an antioxidant that improves glucose uptake by muscle cells. Methods are provided for treating or preventing the complications of diabetes or pre-diabetes, lowering plasma level of HbAlC, and/or lowering glucose plasma levels in a diabetic, pre-diabetic, or non-diabetic mammal while minimizing undesirable side effects.

### BACKGROUND

Diabetes is a chronic, metabolic disease characterized by elevated levels of blood glucose (or blood sugar), which leads over time to serious damage to the heart, blood vessels, eyes, kidneys and nerves. Diabetes is a condition that impairs the body's ability to process blood glucose, otherwise known as blood sugar. High glucose blood levels are responsible for the morbidities and life-style changes associated with diabetes. Type 2 diabetes is a lifelong disease with severe consequences if blood glucose is left uncontrolled. It may lead to irreversible kidney damage (nephropathy), nerve damage (neuropathy), damage to eyes (retinopathy), heart disease and stroke.

Prior to the onset of full-fledged type 2 diabetes, patients undergo a pre-diabetic phase which lasts for years during which there is a year-on-year increase in fasting blood glucose, eventually culminating in full-fledged diabetes if left unchecked.

Normally, muscles take up almost 70% of the glucose from blood which leads to blood glucose lowering. The hormone insulin produced by the islet cells in pancreas is the main trigger for this glucose uptake by muscles. In pre-diabetes and type 2 diabetes, muscles become non-responsive to insulin. Any intervention that can help promote insulin induced muscle glucose uptake will help balance blood glucose levels.

Type 2 diabetes is caused by diet and life style changes such as eating high calorie food, lack of exercise, obesity, family history and high stress being the common causes. In addition high carbohydrate foods lead to high blood sugar. Upon consumption of food, pancreas releases insulin which then triggers muscle taking up glucose and lowering blood sugar. Furthermore, age, stress, sedentary life style, over-eating makes the muscle not responding to insulin (insulin resistance) which leads to high blood glucose and eventually type 2 diabetes.

Once carbohydrate food is ingested, the dietary carbohydrates get broken down in the gut into glucose, which is then absorbed by the intestinal cells into the blood circulation. Within a short time of glucose absorption, the pancreas releases insulin triggering a cascade of steps that clear glucose for circulation. Essentially the steps are that insulin binds to its receptors in various tissues such as muscle, adipose etc. This then recruits the glucose transporters GLUT4 from cytoplasm to the cell membrane where it binds glucose and transports it inside the cells. Once inside the cell's glucose can either be utilized to generate energy or be stored as glycogen. The uptake of glucose by the tissues lowers blood glucose levels.

When blood sugar (glucose) is not controlled, proteins in various organs become glycosylated and become dysfunctional. Some organs are more susceptible to glycosylation such as the kidneys, vascular endothelium, retina, etc., leading to disease states that include reduced wound healing and neuropathy, diabetic retinopathy, chronic kidney disease etc. Controlling blood glucose levels has been shown to have the potential to ameliorate progression of these diseases and other complications of diabetes induced high blood glucose.

While there are medications to control blood glucose in type 2 diabetics, currently there are no interventions prescribed for pre-diabetes. Alternate strategies to control blood glucose are needed either to act alone in pre-diabetes or as supplement to the existing drugs for type 2 diabetes.

FIG. 1 depicts progression from a normal healthy glucose level to a Type 2 diabetes condition. The process of muscle uptake is critical to glucose lowering since muscles account for almost 70% of glucose uptake. During type 2 diabetes, the muscle uptake is defective due to insulin resistance, a process in which muscles do not respond to insulin and therefore glucose uptake is limited.

As depicted in FIG. 1, glucose levels rise in pre-diabetics and type 2 diabetics when insulin is not effective in promoting glucose uptake by muscle. Thus, supplements that help insulin work better can maintain healthy blood glucose.

In a subject with type 2 diabetes, glucose extracted from food in the gut triggers production of insulin by the pancreas. Organs and muscles, however, no longer respond to insulin (insulin resistance), and thus uptake less glucose, resulting in increased blood sugar levels due to insulin resistance.

Thus, controlling blood glucose levels is a desirable therapeutic goal. A number of oral antihyperglycemic agents are known. For example, sulfonylureas and meglitinides increase the insulin output by the pancreas. Thiazolidinediones increase the sensitivity of cells to insulin. Alpha glucosidase inhibitors can decrease the absorption of carbohydrates from the intestine. None of these treatments, however, is ideal. Accordingly, a need exists for new compositions that can be used in the treatment or prevention of pre-diabetes and diabetes in a subject.

While there are medications to control blood glucose in type 2 diabetics, currently there are no interventions prescribed for pre diabetes. Additionally, blood glucose controlling medications control glucose by inducing pancreas to produce insulin or by increasing secretion of glucose in urine or by inducing liver to store glucose as glycogen. However, none of the medications or nutraceuticals or dietary supplements improve glucose utilization by muscle cells, which is the organ that typically utilizes a large majority of blood glucose. Alternate strategies to control blood glucose are needed either to act alone in pre-diabetes or as supplement to the existing drugs for type 2 diabetes. Although there are several medications available to treat type 2 diabetes, they all have limitations and multiple drugs have to be used for maximal control. There is still a need for a safe nutraceutical supplement combination which can act at multiple molecular steps to help insulin action in muscle cells.

### SUMMARY

It is an object of this invention to provide an effective method of preventing or treating diabetes induced high blood glucose levels in a subject while minimizing undesirable side effects. It is also an object of this invention to provide an effective method of preventing or treating pre-diabetes in a subject while minimizing undesirable side effects.

Provided is a nutraceutical composition containing a blend of safe and well tolerated vitamins as a nutraceutical supplement, where the components act synergistically at various steps in the insulin signaling cascade and improve glucose uptake.

The nutraceutical composition provided herein can be used for inhibiting, preventing, mitigating, or treating a pre-diabetic or diabetic condition in a subject. The nutraceutical composition provided herein can be used for treating or preventing the complications of a pre-diabetes or diabetes in a subject. The nutraceutical composition contains a ternary blend of 500 IU of vitamin D3, 12-120 mg of niacinamide, and 50-100 mg of lipoic acid as active ingredients. In some formulations, the vitamin D3, niacinamide, and lipoic acid are the only active ingredients in the composition. The ratio of the vitamin D3 to the niacinamide can be from about 50:1 to 50:12. The ratio of the vitamin D3 to the lipoic acid can be from about 10:1 to 5:1. The ratio of the niacinamide to the lipoic acid can be from about 0.12:1 to 2.4:1. The lipoic acid can be R-alpha-lipoic acid; or the lipoic acid can be S-alpha-lipoic acid; or the lipoic acid can be a blend of R-alpha-lipoic acid and S-alpha-lipoic acid.

The nutraceutical composition provided herein can be in a form selected from among a tablet, a soft gel, a capsule, a pill, a caplet, a dragée, a lozenge, a troche, an effervescent tablet, an emulsion, a chewing gum, a chewable gel product, a wafer, an orally disintegrating tablet, a sublingual tablet, a buccal tablet, a buccal patch, an orodispersible film, granules, a powder, a spray, a solution, a syrup, and a suppository. The nutraceutical composition can include, in addition to the ternary blend of vitamin D3, niacinamide, and lipoic acid, one or a combination of a component selected from among a binder, carrier material, a color agent, a disintegrant, an effervescence agent, an excipient, a filler, a flavor, a lubricant, a surfactant, a suspending agent, and a sweetener.

Also provided is method of treating or preventing pre-diabetes in a subject in need thereof. The method includes administering to the patient a therapeutically effective amount of the nutraceutical composition provided herein, wherein after 12 weeks of administration an average decrease of a fasting blood glucose level can be 20% or more based on fasting plasma glucose level prior to administration. In some methods, the fasting blood glucose level can be reduced to less than 100 mg/dL after 25 weeks, or 20 weeks, or 15 weeks, or 10 weeks of administration.

Also provided is a method of treating or preventing diabetes mellitus in a subject in need thereof. The method includes administering to the patient a therapeutically effective amount of the nutraceutical composition provided herein; and a therapeutically effective amount of an agent selected from the group consisting of insulin, a meglitinide, a thiazolidinedione, an alpha glucosidase inhibitor, a dipeptidyl peptidase-4 (DPP-4) inhibitor, a biguanide, a SGLT2 inhibitor, a GLP agonist, an incretin mimetic, a dopamine-2 agonist, a bile acid sequestrant, and sulfonylurea, where an HbA1C level, as measured after 12 weeks of administration, is reduced in the patient. In the method, the dipeptidyl peptidase-4 (DPP-4) inhibitor can be sitagliptin. In the method, the biguanide can be metformin. In the method, the SGLT2 inhibitor can be empagliflozin. In the method, the incretin mimetic inhibitor is semaglutide. In the method, the subject can be non-insulin dependent. In the method, the HbA1C level can be reduced by at least 0.45 percentage point.

Also provided is a method of lowering blood glucose level in an animal that is diabetic, non-diabetic, or pre-diabetic, the method including administering to the animal a therapeutically effective amount of the nutraceutical composition provided herein, where a fasting blood glucose level can be reduced in the animal to less than 100 mg/dL after 25 weeks, or 20 week, or 15 weeks, or 10 weeks of administration.

Also provided is a method of lowering a plasma level of HbA1C in an animal that is diabetic, non-diabetic, or pre-diabetic, the method including administering to the animal a therapeutically effective amount of the nutraceutical composition provided herein, where the plasma level of HbA1C can be reduced in the animal by at least 0.25 percentage point after 10 weeks of administration. The animal can be a mammal, or the animal can be a non-human mammal.

Also provided is a method for delaying onset of or preventing diabetic retinopathy in a subject that is diabetic, non-diabetic, or pre-diabetic, the method including administering to the subject a therapeutically effective amount of the nutraceutical composition provided herein, where the HbA1C level can be reduced by at least 0.45 percentage point after 12 weeks of treatment. Also provided is a method for delaying onset of or preventing diabetic neuropathy in a subject that is diabetic, non-diabetic, or pre-diabetic, the method including administering to the subject a therapeutically effective amount of the nutraceutical composition provided herein, where the HbA1C level can be reduced by at least 0.45 percentage point after 12 weeks of treatment. Also provided is a method for delaying onset of or preventing diabetic nephropathy in a subject that is diabetic, non-diabetic, or pre-diabetic, the method including administering to the subject a therapeutically effective amount of the nutraceutical composition provided herein, wherein the HbA1C level is reduced by at least 0.45 percentage point after 12 weeks of treatment.

In the methods provided herein, the nutraceutical composition can be administered by oral, intravenous, intramuscular, intraperitoneal, subcutaneous, rectal, mucosal, to transdermal administration. The nutraceutical composition can be administered in a dosage form selected from among a tablet, a soft gel, a capsule, a pill, a caplet, a dragée, a lozenge, a troche, an effervescent tablet, an emulsion, a chewing gum, a chewable gel or gummy, a wafer, an orally disintegrating tablet, a sublingual tablet, a buccal tablet, a buccal patch, an orodispersible film, granules, a powder, a spray, a solution, a syrup, and a suppository. In the methods, the nutraceutical composition can be administered once a day. The nutraceutical composition can be administered before, during, or after consuming the largest meal of the day.

Also provided herein is the use of the nutraceutical compositions provided herein as a medicament for the treatment of pre-diabetes. Also provided herein is the use of the nutraceutical compositions provided herein as a medicament for the treatment of diabetes.

The nutraceutical compositions provided herein can be used as a medicament for the treatment of diabetes or pre-diabetes. The nutraceutical composition provided herein can be used in combination with another therapeutic agent selected from the group consisting of insulin, a meglitinide, a thiazolidinedione, an alpha glucosidase inhibitor, a dipeptidyl peptidase-4 (DPP-4) inhibitor, a biguanide, a SGLT2 inhibitor, a GLP agonist, an incretin mimetic, a dopamine-2 agonist, a bile acid sequestrant, and sulfonylurea. The nutraceutical composition provided herein can be used for reducing an HbA1C level in a subject that is diabetic, non-diabetic, or pre-diabetic. The nutraceutical composition provided herein can be used for reducing a fasting blood glucose level in a subject that is diabetic, non-diabetic, or pre-diabetic. The nutraceutical composition provided herein can be used for delaying onset of or preventing diabetic retinopathy or diabetic neuropathy or diabetic nephropathy in a subject that is diabetic, non-diabetic, or pre-diabetic.

Also provided are articles of manufacture, which include the nutraceutical composition provided herein and a pharmaceutical packaging material selected from among blister packs, bottles, tubes, inhalers, pumps, bags, vials, containers, syringes, bottles, and combinations thereof. Also provided are kits. The kit can include the nutraceutical composition provided herein and one or a combination of instructions for dosages, instructions for dosing regimens, and instructions for modes of administration. The kit can include the nutraceutical composition provided herein and a device for determining blood glucose levels.

### BRIEF DESCRIPTION OF THE DRAWINGS

FIG. 1 is a schematic representation showing the progression from healthy glucose levels to Type 2 diabetes

FIG. 2 is a schematic showing glucose regulation as mediated by insulin uptake by muscle.

FIG. 3 shows a depiction of the receptors on a muscle cell membrane to illustrate how the composition of the present invention in muscle cells participates in controlling glucose. Vitamin D3 increases the expression of GLUT4, which allows more intake of glucose by insulin. Niacinamide promotes glucose utilization by the muscle. Lipoic acid works as an antioxidant to improve mitochondrial energy generation.

FIG. 4 shows a graphical comparison of the effects of vitamin D3 and niacinamide separately and a combination of vitamin D3 and niacinamide on glucose uptake by muscle cells in laboratory studies. ***=P<0.001; **=P<0.01; and *=P<0.05.

FIG. 5 shows a graphical comparison glucose uptake by muscle cells in laboratory studies. V=Vitamin D at 500 IU. N=Niacinamide at 15 mg/ml. LA=Lipoic Acid at 0.5 mM. The data shown are statistically significant for the V+N and LA+V+N from control, p>0.05.

FIG. 6 shows a graph of fasting blood glucose values in a subject administered a nutraceutical composition provided herein for 25 weeks.

FIG. 7 shows a graph for the HbA1C profile in a subject administered a nutraceutical composition provided herein for 25 weeks.

### EXPLANATION OF REFERENCE NUMBERS

10: outside cell membrane
15: cell membrane
20: inside cell membrane
100: insulin
200: inactive insulin receptors
250: active insulin receptors
300: glucose
400: GLUT4 glucose transporter

### DETAILED DESCRIPTION

Hereinafter, embodiments of the present disclosure will be described in more detail to assist in the understanding of the invention.

For clarity of disclosure, and not by way of limitation, the detailed description is divided into the subsections that follow.

Further aspects, features and advantages of this invention will become apparent from the detailed description which follows. It should be understood that the various individual aspects and features of the present invention described herein can be combined with any one or more individual aspect or feature, in any number, to form embodiments of the present invention that are specifically contemplated and encompassed by the present invention. Furthermore, any of the features recited in the claims can be combined with any of the other features recited in the claims, in any number or in any combination thereof. Such combinations are also expressly contemplated as being encompassed by the present invention.

### Definitions

Unless defined otherwise, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the singular forms “a,” “an” and “the” include plural referents unless the context clearly dictates otherwise.

As used herein, all ranges include the upper and lower limits. As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the end-points of the range. As an example, a variable which is described as having values between 0 and 10, can be 0, 4, 2-6, 2.75, 3.3-4.4, etc.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics. All of the values characterized by the above-described modifier “about,” are also intended to include the exact numerical values associated therewith. Hence “about 5 percent” means “about 5 percent” and also “5 percent.”

As used herein, “optional” or “optionally” means that the subsequently described event or circumstance does or does not occur, and that the description includes instances where said event or circumstance occurs and instances where it does not. For example, an optional component in a system means that the component may be present or may not be present in the system.

As used herein, the terms “comprises” and “comprising” are inclusive and open ended, and not exclusive. When used in the specification and claims, the terms “comprises” and “comprising” and variations thereof mean the specified features, steps or components are included, but do not exclude other features, steps or components.

Any compositions described herein are intended to encompass compositions which consist of, consist essentially of, as well as comprise, the various constituents identified herein, unless explicitly indicated to the contrary.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

As used herein, the term “exemplary” means “serving as an example or illustration,” and should not be construed as being preferred or advantageous over other configurations disclosed herein.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

As used herein, the term “subject” and “patient” includes members of the animal kingdom including but not limited to human beings.

As used herein, the term “animal” includes any animal, such as, but not limited to, primates including humans, gorillas and monkeys; rodents, such as mice and rats; fowl, such as chickens or turkeys; ruminants, such as goats, cows, deer, and sheep; a porcine; felines; canines; and other animals. Non-human animals exclude humans as the contemplated animal.

As used herein, the term “wt % based on the weight of the composition” refers to mass % or (w/w) %.

As used herein, “disease or disorder” refers to a pathological condition in an organism resulting from a cause or condition including, but not limited to, increased bold glucose levels and/or increased HbA1C levels, and that is characterized by identifiable symptoms.

As used herein, “treating” a subject with a disease or condition means that the subject's symptoms are partially or totally alleviated, or remain static following treatment.

As used herein, treatment refers to any effects that ameliorate symptoms of a disease or disorder. Treatment encompasses prophylaxis, therapy and/or cure. Treatment also encompasses any pharmaceutical use of any composition provided herein.

As used herein, prophylaxis refers to prevention of a potential disease and/or a prevention of worsening of symptoms or progression of a disease.

As used herein, “prevention” or prophylaxis, and grammatically equivalent forms thereof, refers to methods in which the risk or probability of developing a disease or condition is reduced.

As used herein, a “pharmaceutically effective agent” includes any therapeutic agent or bioactive agents, that exhibit a therapeutic effect.

As used herein, a “therapeutic effect” means an effect resulting from treatment of a subject that alters, typically improves or ameliorates, the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, “therapeutic efficacy” refers to the ability of an agent, compound, material, or composition containing a compound to produce a therapeutic effect in a subject to whom the agent, compound, material, or composition containing a compound has been administered.

As used herein, a “prophylactically effective amount” or a “prophylactically effective dose” refers to the quantity of an agent, compound, material, or composition containing a compound that when administered to a subject, will have the intended prophylactic effect, e.g., preventing or delaying the onset, or reoccurrence, of disease or symptoms, reducing the likelihood of the onset, or reoccurrence, of disease or symptoms. The full prophylactic effect does not necessarily occur by administration of one dose, and can occur only after administration of a series of doses. Thus, a prophylactically effective amount can be administered in one or more administrations.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, a “combination” refers to any association between or among two or more items. The combination can be two or more separate items, such as two compositions or two collections, a mixture thereof, such as a single mixture of the two or more items, or any variation thereof. The elements of a combination are generally functionally associated or related.

As used herein, combination therapy refers to administration of two or more different therapeutics. The different therapeutic agents can be provided and administered separately, sequentially, intermittently, or can be provided in a single composition.

As used herein, a kit is a packaged combination that optionally includes other elements, such as additional reagents and instructions for use of the combination or elements thereof, for a purpose including, but not limited to, activation, administration, diagnosis, and assessment of a biological activity or property.

As used herein, a “unit dose form” refers to physically discrete units suitable as unitary dosages for human and animal subjects and packaged individually as is known in the art. Each unit contains a predetermined quantity of active material calculated to produce the desired therapeutic effect, in association with any suitable pharmaceutical excipient (e.g., a tablet or capsule).

As used herein, a “single dosage formulation” refers to a formulation for direct administration.

As used herein, an “article of manufacture” is a product that is made and sold. As used throughout this application, the term is intended to encompass any of the compositions provided herein contained in articles of packaging.

As used herein, a “nutraceutical composition” refers to a composition containing a pharmaceutical grade or standardized nutrient that provides a health or medical benefit, including the prevention and/or treatment of a disease. An exemplary nutraceutical composition is a dietary supplement.

As used herein, “lipoic acid” refers to alpha-lipoic acid, CAS Number 1077-28-7. The lipoic acid can be either of the optical isomer R- and S-forms of alpha-lipoic acid, or a combination thereof.

As used herein, a “beverage,” refers to an aqueous formulation suitable for oral consumption by a subject.

As used herein, “diabetes” refers to “diabetes mellitus” and is a disease state characterized by hyperglycemia; altered metabolism of lipids, carbohydrates, and proteins; and an increased risk of complications from vascular disease. The fasting blood sugar level in a diabetic subject typically is 126 mg/dL or higher, or 130 mg/dL or higher.

As used herein, “pre-diabetes” refers to a condition where the subject consistently has a fasting blood glucose that is between 100 to about 125 mg/dL.

As used herein, “HbA1C” refers to glycolated hemoglobin. Hemoglobin undergoes glycosylation on its amino terminal valine residue to form the glucosyl valine adduct of hemoglobin (HbA1C). The toxic effects of hyperglycemia may be the result of accumulation of such nonenzymatically glycosylated products. The covalent reaction of glucose with hemoglobin also provides a convenient method to determine an integrated index of the glycemic state. For example, the half-life of the modified hemoglobin is about 120 days. Since the amount of glycosylated protein is proportional to the glucose concentration and the time of exposure of the protein to glucose, the concentration of HbA1C in the circulation reflects the glycemic state over an extended period (4 to 12 weeks) prior to sampling.

As used herein, “FDC” refers to the fixed dose combination of vitamin D3, niacinamide and lipoic acid provided herein.

### Muscle Uptake of Glucose

Once carbohydrate food is ingested, the dietary carbohydrates get broken down in the gut into glucose, which is then absorbed by the intestinal cells and transported into circulating blood. Within a short time of glucose absorption, the pancreas releases insulin, triggering a cascade of steps that clear glucose for circulation. Essentially the steps are that insulin binds to its receptors in various tissues such as muscle, adipose etc. This then recruits the glucose transporters GLUT4 from cytoplasm to the cell membrane where the transporters bind glucose and transport it inside the cells. Once inside the cell, glucose can either be utilized to generate energy or be stored as glycogen. The uptake of glucose by the tissues lowers blood glucose levels.

The various molecular steps in glucose uptake by muscle are:
1. Insulin triggers the uptake of glucose into cells by triggering relocation of GLUT4 transport protein
2. The glucose is then used in the mitochondria to generate energy through a series of redox reactions which utilize nicotinamide
3. The redox reactions generate oxidative stress which if not controlled can inhibit ATP generation

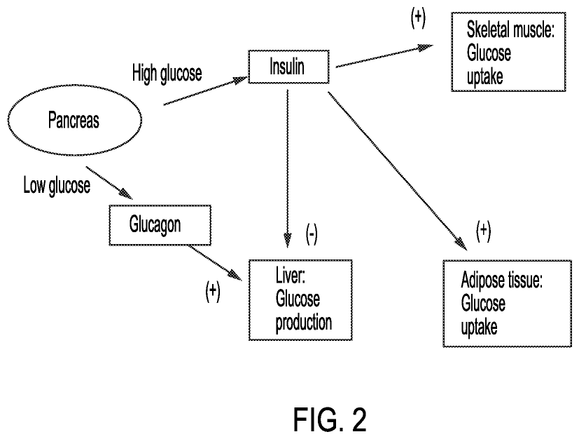

The process of muscle uptake is critical to glucose lowering since muscles account for almost 70% of glucose uptake. During type 2 diabetes, the muscle uptake is defective due to insulin resistance, a process in which muscles do not respond to insulin and therefore glucose uptake is limited (FIG. 2). As shown in FIG. 2, high blood glucose triggers secretion of insulin by the pancreas. The insulin allows muscles to take up glucose and lower blood glucose levels. When the muscles do not respond to insulin, the glucose is taken up by adipose tissue, increasing the amount of fat and body weight in a subject. The compositions provided herein improve muscle glucose update and help to maintain better blood glucose levels and result in better glucose control. The compositions provided herein have therapeutic efficacy of improving muscle glucose update and helping to maintain better blood glucose levels in a subject to whom the composition has been administered.

Type 2 Diabetes is a condition that impairs the body's ability to process blood glucose. High glucose blood levels are responsible for the morbidities and life-style changes associated with diabetes. Type 2 diabetes is a lifelong disease with severe consequences if blood glucose is left uncontrolled. It can lead to irreversible kidney damage (nephropathy), nerve damage (neuropathy), damage to eyes (retinopathy), heart disease, and stroke.

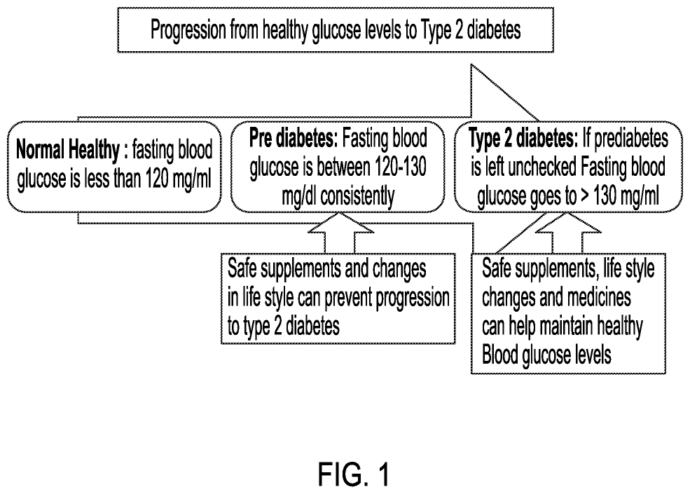

Prior to the onset of full-fledged type 2 diabetes, patients undergo a pre-diabetic phase which can last for years, during which there is a year-on-year increase in fasting blood glucose, eventually culminating in full-fledged diabetes if left unchecked. FIG. 1 shows the typical progression from normal blood glucose level to full-fledged type 2 diabetes.

Type 2 diabetes is caused by diet and life style changes such as eating high calorie food, lack of exercise, obesity, family history and high stress being the common causes. In addition high carbohydrate foods lead to high blood sugar. Upon consumption of food, pancreas releases insulin which then triggers muscle taking up glucose and lowering blood sugar. Furthermore, age, stress, sedentary life style, over-eating makes the muscle not responding to insulin (insulin resistance) which leads to high blood glucose and eventually type 2 diabetes (Kahn et al., Lancet 383(9922): 1068-1083 (2014); Chang et al., Molecular Medicine 10(7-12): 65-71 (2004)).

Although there are several medications available to treat type 2 diabetes, they all have limitations and multiple drugs have to be used for maximum control. There is still a need for a safe nutraceutical supplement combination which can act at multiple molecular steps to help insulin action in muscle cells. To this end we have developed a mix of safe and well tolerated vitamins as nutraceutical supplement which act synergistically at various steps in the insulin signalling cascade and improve glucose uptake.

Normally, muscles take up almost 70% of the glucose from blood which leads to blood glucose lowering. The hormone insulin produced by the islet cells in pancreas is the main trigger for this glucose uptake by muscles. In pre-diabetes and type 2 diabetes, muscles become non-responsive to insulin (Kahn et al., Lancet 383(9922): 1068-1083 (2014); Chang et al., Molecular Medicine 10(7-12): 65-71 (2004)). Any intervention that can help promote insulin-induced muscle glucose uptake will help balance blood glucose levels (FIG. 2 below shows glucose uptake pathway).

While there are many drugs to treat type 2 diabetes by lowering blood glucose, researchers continue to develop novel ways to manage diabetes. Current drugs (e.g., metformin, JANUVIA® (sitagliptin), SGLT2 inhibitors, GLP agonists, sulfonylurea, etc.) act on organ systems such as the pancreas, liver, kidneys, GI tract etc. to help manage blood glucose, but not muscle, which is the largest blood glucose eliminating organ in the body. The nutraceutical composition provided herein promotes the utilization of blood glucose by the muscle. Given the need for safe and effective interventions, we explored the use of targeted vitamins which can improve muscle glucose uptake. In addition to traditional drug development, the researchers have also been focused on health supplements to augment other therapeutic strategies to lower blood glucose levels. Use of supplements alongside diabetes medication can enable the diabetic patient to decrease the dosage of traditional diabetes medication dose. In addition, pre-diabetics can hope to prevent onset of full-fledged type diabetes. With this in mind applicant has designed and developed a novel combination of safe and approved vitamins to combat high blood glucose levels.

The composition provided herein, which includes two vitamins with established safety and at levels that have been deemed safe in humans and that have used for decades or longer, and lipoic acid, an antioxidant. The combination improves the uptake of glucose and the metabolization of glucose to generate energy. The composition is an alternative novel intervention of great value for glucose control in pre diabetics and can be used as adjunct supplement in the treatment of subjects having type 2 diabetes. The composition allows the HbA1C and blood glucose levels to be controlled, which can result in the risk of complications such as cardiovascular disease, retinopathy, nephropathy, and neuropathy to be reduced or their onset delayed. The compositions herein can be administered in a prophylactically effective amount to have the intended prophylactic effect of controlling HbA1C and blood glucose levels.

### Compositions

The present invention provides a nutraceutical composition for administration to subjects with pre-diabetes or type 2 diabetes that improves glucose uptake by muscle cells. The composition is a fixed dose combination of vitamin D3, niacinamide, a form of vitamin B3, and lipoic acid, an antioxidant. The composition can be provided as a health supplement.

The present invention provides a composition that is a combination and is backed by several scientific and clinical independent studies that demonstrate that the individual components vitamin D3, niacinamide and lipoic acid when consumed can act as potential suppressing agents of high blood glucose levels. A review of clinical studies in people with type 2 diabetes observed that taking Vitamin D 3 in combination with diet and lifestyle changes reduced fasting blood sugar and HBA1C compared to diet and lifestyle changes alone or in combination with a placebo. Similarly, preclinical and clinical data have shown niacinamide also helps in glucose control. Finally lipoic acid has been used to control blood glucose levels.

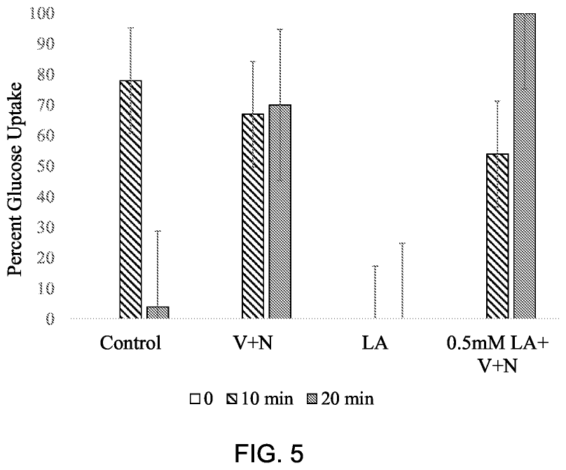

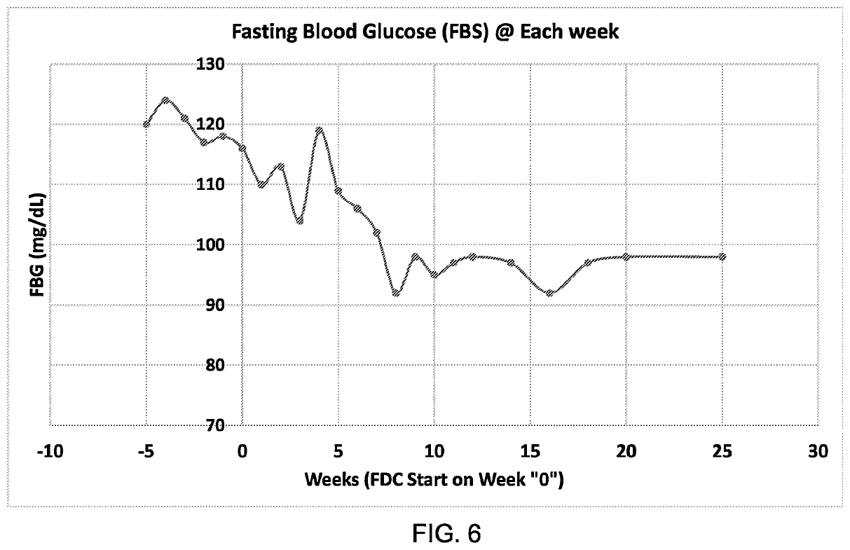

Applicant herein presents various concentrations and combination ratios of vitamin D3 and niacinamide in combination with lipoic acid shown to synergistically interact resulting in promoting glucose uptake by muscle cells in response to insulin. It has been found that the combination is superior in promoting glucose uptake by muscle cells due to a unique synergistic TRIPLE ACTION (see FIGS. 5 and 6). Based on these observations, provided is a unique TRIPLE ACTION combination of vitamin D3, niacinamide, and lipoic acid that is optimal for blood glucose lowering, and forms the basis for the novel combination. The combination exhibits a synergistic interaction that achieves more effective plasma glucose and HbAlC reductions than can be achieved by any of the components on their own. The combination improves glucose uptake by muscle cells.

The compositions provided herein include 500 IU of vitamin D3 (cholecalciferol, CAS Number: 67-97-0), 12-120 mg of niacinamide (CAS Number 98-92-0), and 50-100 mg of DL-alpha-lipoic acid (CAS Number 1077-28-7). In the composition, the ratio of vitamin D3 to niacinamide can be from about 50:1 to 50:12. In the composition, the ratio of vitamin D3 to alpha-lipoic acid can be from about 10:1 to 5:1.

The nutraceutical/pharmaceutical composition of the invention provided herein containing a ternary blend of 500 IU of vitamin D3, 12-120 mg of niacinamide, and 50-100 mg of lipoic acid as active ingredients. The recited ranges of vitamin D3 and niacinamide can be equal to any value or values within any of the above-described numerical ranges, including the end-points of these ranges. For example, the niacinamide can be present in an amount of 12-120 mg, 15-100 mg, 12-20 mg, 100-120 mg., 12.5 to 55.56 mg, or 12.0 to 22.5 mg. In some formulations, the niacinamide can be present in an amount of at least 12 mg, at least 15 mg, at least 20 mg, at least 25 mg, at least 50 mg, at least 75 mg, at least 90 mg, or at least 100 mg. The niacinamide can be present in an amount of up to 120 mg, or up to 100 mg, or up to 75 mg, or up to 50 mg, or up to 25 mg, or up to 15 mg. The lipoic acid can be present in an amount of 50-100 mg, or 55-95 mg, or 60-80 mg, or 50-75 mg, or 50.5-99.5 mg. The lipoic acid can be present in an amount of at least 50 mg, at least 55 mg, at least 60 mg, at least 65 mg, at least 70 mg, at least 75 mg, at least 80 mg, at least 85 mg, at least 90 mg, or at least 95 mg. The lipoic acid can be present in an amount of up to 100 mg, or up to 90 mg, or up to 80 mg, or up to 70 mg, or up to 60 mg.

The nutraceutical/pharmaceutical composition of the invention provided herein can contain a ternary blend of 500 IU of vitamin D3, 12-120 mg of niacinamide, and 50-100 mg of lipoic acid as the only active ingredients.

### Forms of the Compositions

The nutraceutical/pharmaceutical composition of the invention provided herein containing a ternary blend of 500 IU of vitamin D3, 12-120 mg of niacinamide, and 50-100 mg of lipoic acid as active ingredients can be provided in a form appropriate for the desired route of administration. For example, the composition can be administered in a form appropriate for oral, parenteral (intravenous, intramuscular, intraperitoneal, intrasternal, subcutaneous, intraarticular injection and infusion), percutaneous, rectal, mucosal, or topical (transdermal, as by powders, ointments, creams, sprays, drops or patches) administration. The composition can be provided in a form selected from among a tablet, a soft gel, a capsule, a pill, a caplet, a dragée, a lozenge, a troche, an effervescent tablet, an emulsion, a chewing gum, a chewable gel or gummy, a wafer, an orally disintegrating tablet, a sublingual tablet, a buccal tablet, a buccal patch, an orodispersible film, granules, a powder, a spray, a solution, a syrup, and a suppository. The form of the composition can include, in addition to the ternary blend of active ingredients, one or a combination of a binder, carrier material, a color agent, a disintegrant, an effervescence agent, an excipient, a filler, a flavor, a lubricant, a surfactant, a suspending agent, and a sweetener. In some embodiments, the pharmaceutical composition is in the form of pellets, beads, granules or minitablets in a capsule. In some embodiments, the pharmaceutical composition is in the form of pellets, beads, or granules that are compressed into a single tablet.

A solid nutraceutical/pharmaceutical composition for oral administration can optionally contain, in addition to the ternary blend of active ingredients, carrier materials such as gum arabic, corn starch, gelatin, dextrin, maltodextrin, cyclodextrin, microcrystalline cellulose, kaolin, dicalcium phosphate, calcium carbonate, sodium chloride, alginic acid or a salt thereof, xanthan gum, gellan gum, and the like; disintegrators including, microcrystalline cellulose, alginic acid or a salt thereof, cross-linked polyvinyl pyrrolidinone, guar gum, and the like; binders including gum arabic, methylcellulose, ethyl cellulose, sodium carboxymethylcellulose, polyvinylpyrrolidone, hydroxypropyl methylcellulose, and the like; and lubricants such as magnesium stearates, stearic acid, silicone fluid, talc, oils, waxes, colloidal silica, and the like.

A solid nutraceutical/pharmaceutical composition for oral administration also can be provided to contain an effervescence agent to produce an effervescent tablet. In such dosage forms, in addition to active ingredients, the effervescent tablet composition can contain mixtures of acids (such as citric acid, fumaric acid, lactic acid, malic acid, tartaric acid, or a combination thereof) and carbonates like sodium, potassium bicarbonate or carbonate that release carbon dioxide when dissolved in water or an aqueous bodily fluid. An effervescent tablet can include a water soluble binder (such as starches, modified starches, natural gums, cellulose gums, microcrystalline cellulose, methylcellulose, cellulose ethers, ethylcellulose, sodium carboxymethylcellulose, gelatin, polyethylene glycol, polyvinylpyrrolidone, pectins, alginates, xanthan gum, gellan gum, polyacrylamides, polyvinyloxoazolidone, polyvinylalcohols and mixtures thereof) and lubricant (sodium benzoate, polyethylene glycol, L-leucine, adipic acid, and combinations thereof).

A solid form of the nutraceutical/pharmaceutical composition for oral administration composition optionally can include one or more coatings. The coatings can be provided to modify or improve the physical integrity and/or appearance of the solid dosage form of the composition. The coating can be a controlled release coating, or a moisture barrier coating, or an enteric coating, or any combination thereof.

In a preferred form, provided is a dietary supplement in which the composition provided herein is provided in a capsule. The capsules are prepared by providing blending the components of the composition together to form a uniform blend, and filling a soft gelatin or hard gelatin or vegetable capsules with the uniform blend to produce capsules for oral administration. Fillers and carriers can be blended with the blend of vitamin D3, niacinamide, and lipoic acid to prepare the filling for the capsules. The blend also can be packaged into a sachet packet to deliver the composition in the form of a flowable powder, that can be sprinkled on food or dissolved into a beverage or water. The composition containing the blend of vitamin D3, niacinamide, and lipoic acid also can be provided as a unit dosage in the form of a compressed tablet using methods well known in the pharmaceutical arts.

The compositions of the invention can be formulated to provide the desired release profile to provide quick, sustained or delayed release of the active ingredients after administration to the patient by employing procedures known in the art. Controlled release drug delivery systems for oral administration include osmotic pump systems and dissolution systems containing polymer-coated reservoirs or drug-polymer matrix formulations. Examples of controlled release systems are described in U.S. Pat. No. 3,845,770 (Theeuwes et al., 1974); U.S. Pat. No. 4,326,525 (Swanson et al., 1982); U.S. Pat. No. 4,902,514 (Barclay et al., 1990); and U.S. Pat. No. 5,616,345 (Geoghegan et al., 1997).

The nutraceutical/pharmaceutical composition containing the blend of vitamin D3, niacinamide, and lipoic acid also can be provided in the form of a liquid for oral administration. Various liquid and powder nutraceutical compositions can be prepared by conventional methods. The liquid nutraceutical/pharmaceutical compositions can be in the form of a solution, emulsion, syrup, gel, or elixir. The liquid can contain the blend dissolved or suspended in water or any other aqueous vehicle. In addition to the above enumerated nutraceutical/pharmaceutical composition ingredients or compounds, a liquid nutraceutical/pharmaceutical composition can include suspending agents such as, for example, alginates, pectin, xanthan gum, gellan gum, carrageenan, gum arabic, methylcellulose, polyvinyl alcohol, polyvinylpyrrolidone, and the like. The liquid forms also can include one or more wetting agents, sweeteners, and coloring and flavoring agents.

The nutraceutical/pharmaceutical composition containing the blend of vitamin D3, niacinamide, and lipoic acid also can be provided in the form of an orodispersible film for oral administration. Orodispersible films are thin films that dissolve in the mouth (see Cupone et al., Pharmaceutics 2022, 14, 2011, https://doi.org/10.3390/pharmaceutic14102011, 17 pages). Such films rapidly dissolve in the mouth and do not need to be swallowed. The orodispersible films can contain a polymer, a plasticizer, the blend of vitamin D3, niacinamide, and lipoic acid, and optionally surfactants, flavors, or taste-masking agents. The polymer can be one or a combination of carboxymethyl cellulose, hydroxypropyl cellulose, polyvinyl pyrrolidone, chitosan, pullulan, gelatin, gellan gum, maltodextrin, sodium alginate, starch, modified starch, xanthan gum, and locust bean gum. Exemplary plasticizers include glycerin, olive oil, vegetable oil, propylene glycol, mannitol, sorbitol, triacetate, and citrate ether. A comprehensive review of technologies to fabricate orodispersible gels is available (Gupta et al., Pharmaceutics 2022, 14, 820, https://doi.org/10.3390/pharmaceutic14040820, 30 pages).

The nutraceutical/pharmaceutical composition containing the blend of vitamin D3, niacinamide, and lipoic acid also can be provided in the form of a gummy or chewable gel product. The chewable gel product can contain the blend of vitamin D3, niacinamide, and lipoic acid, one or more gelling agents, one or more pH adjusting agents, one or more wetting agents, and as optional components, one or more sweeteners, one or more flavor agents, or one or more colorants. In some embodiments, the chewable gel product can formulated to provide a texture similar to gummy candies (e.g., gummy bears). Such chewable gel products can be suitable for oral administration to subjects with dysphagia.

Suitable gelling agents for use in the chewable gel product include agar, alginic acid or salts thereof, carrageenan (kappa carrageenan, lambda carrageenan, iota carrageenan), gelatin, gellan gum, high-methoxyl pectin, low-methoxyl pectin, modified food starch, carrageenan, locust bean gum, xanthan gum, and combinations thereof. Cations can be used to modify the resulting texture of the chewable gel product depending on the gelling agent(s) used. Suitable cations include, without limitation, calcium, magnesium, sodium, potassium, and combinations thereof. When present, the cations in the chewable gel product can be present in an amount from about 0.05 wt % to about 1 wt % based on the total weight of the chewable gel product.

The nutraceutical/pharmaceutical compositions for parenteral and percutaneous administration can include pharmaceutically acceptable sterile aqueous or nonaqueous solutions, dispersions, suspensions or emulsions and sterile powders for reconstitution into sterile injectable solutions or dispersions that contain the blend of vitamin D3, niacinamide, and lipoic acid. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Further, the injections may contain stabilizers, solubilizers, suspending agents, emulsifiers, soothing agents, buffers, preservatives, isotonic agents, antibacterial and antifungal agents, etc. The injectable compositions can be sterilized in the final formulation step or prepared by sterile procedures. The nutraceutical/pharmaceutical composition of the invention may also be formulated into a sterile solid preparation, for example, by freeze-drying, and the sterile solid preparation can be dissolved in a solvent, sterile injectable water, injectable saline or other sterile diluent(s), to form an injectable solution for immediate injection.

A pharmaceutical composition solution for parenteral administration also can be prepared by dissolving the blend of vitamin D3, niacinamide, and lipoic acid in a medium, subjecting the resulting solution to filtration for sterilization, filling the solution in vials or ampoules or glass bottles or suitable plastic bags, and sealing the vials or ampoules or bottles or bags. It is also possible to freeze-dry the composition and fill the resulted powder in vials, and then eliminate the moisture in vacuum to improve stability. Parenteral suspensions can be prepared by substantially the same method as that applied to solutions for parenteral administration; however, the suspensions can preferably be manufactured by suspending the active ingredient in a medium, and then subjecting the result to sterilization by using ethylene oxide or the like. Furthermore, surface active agents, wetting agents and so forth can be added so that a uniform dispersion of the active ingredient can be obtained.

Compositions for rectal administration can be provided in the form of suppositories that can be prepared by mixing the blend of vitamin D3, niacinamide, and lipoic acid with suitable non-irritating carriers such as cocoa butter, polyethylene glycol or a suppository wax which are solid at ambient temperature but liquid at body temperature and therefore melt in the rectum and release the blend of active compounds.

Compositions for topical or transdermal administration of composition containing the blend of vitamin D3, niacinamide, and lipoic acid can be prepared as ointments, pastes, creams, lotions, gels, powders, solutions, sprays, inhalants or patches. The blend of active components is admixed under sterile conditions with a pharmaceutically acceptable carrier and any needed preservatives or buffers as may be required. The ointments, pastes, creams and gels can contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof. Transdermal patches can be used to provide continuous or discontinuous infusion of the compounds of the present invention in controlled amounts. The construction and use of transdermal patches for the delivery of pharmaceutical agents is well known in the art. See, e.g., U.S. Pat. No. 4,992,445 (Lawter et al., 1991); U.S. Pat. No. 5,001,139 (Lawter et al., 1991); and U.S. Pat. No. 5,023,252 (Hseih, 1991). Such patches may be constructed for continuous, pulsatile, or on demand delivery of pharmaceutical agents.

### Packaging and Articles of Manufacture

Also provided are articles of manufacture containing packaging materials, any nutraceutical/pharmaceutical composition provided herein, and a label that indicates that the compositions are to be used for treatment of diseases or conditions as described herein. For example, the label can indicate that the treatment is for pre-diabetes or diabetes.

The nutraceutical/pharmaceutical composition provided herein described herein containing the blend of vitamin D3, niacinamide, and lipoic acid, optionally in combination with another therapeutic agent, also can be packaged in an article of manufacture. In one example, the article of manufacture contains a pharmaceutical composition containing the containing the blend of vitamin D3, niacinamide, and lipoic acid and no further agent or treatment. In other examples, the article of manufacture contains another further therapeutic agent, such as insulin, a meglitinide to increase the insulin output by the pancreas, a thiazolidinedione to increase the sensitivity of cells to insulin, an alpha glucosidase inhibitors to decrease the absorption of carbohydrates from the intestine, a sitagliptin (dipeptidyl peptidase-4 (DPP-4) inhibitor, such as Januvia®), a biguanide (such as metformin), a SGLT2 inhibitor, a GLP agonist, an incretin mimetic, a dopamine-2 agonist, a bile acid sequestrant, sulfonylurea, or a combination thereof. The further therapeutic agents can be provided together or separately, for packaging as articles of manufacture.

The articles of manufacture provided herein contain packaging materials. Packaging materials for use in packaging pharmaceutical products are well known to those of skill in the art. See, for example, U.S. Pat. No. 5,033,252 (Carter, 1991); U.S. Pat. No. 5,323,907 (Kalvelage. 1994); and U.S. Pat. No. 5,052,558 (Carter, 1991). Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. Exemplary of articles of manufacture are containers including the pharmaceutical compositions provided herein. The containers include, but are not limited to, tubes, bottles and syringes. The containers can further include a needle for intravenous administration.

The choice of package depends on the agents, and whether such compositions will be packaged together or separately. In general, the packaging is non-reactive with the compositions contained therein. In other examples, some of the components can be packaged as a mixture. In other examples, all components are packaged separately. Thus, for example, the components can be packaged as separate compositions that, upon mixing just prior to administration, can be directly administered together. Alternatively, the components can be packaged as separate compositions for administration separately.

Selected compositions including articles of manufacture thereof also can be provided as kits. Kits can include a nutraceutical/pharmaceutical composition described herein and an item for administration provided as an article of manufacture. The compositions can be contained in the item for administration or can be provided separately to be added later. The kit can, optionally, include instructions for application including dosages, dosing regimens and instructions for modes of administration. Kits also can include a pharmaceutical composition described herein and an item for diagnosis, such as a device for determining blood glucose levels. For example, the kit can include the nutraceutical/pharmaceutical composition described herein, and a glucose test strip, a glucose meter, a continuous glucose monitor, or a noninvasive blood glucose detection device.

### Methods of Treatment and Uses

The methods provided herein include methods of administering or using the vitamin D3, niacinamide, and lipoic acid, for treating subjects having a disease or condition whose symptoms can be ameliorated or lessened by administration of the blend of vitamin D3, niacinamide, and lipoic acid to lower blood glucose levels and/or HbA1C levels.

In particular examples, the disease or condition is pre-diabetes or diabetes. Additionally, methods of combination therapies with one or more additional agents for treatment, such as insulin, a meglitinide to increase the insulin output by the pancreas, a thiazolidinedione to increase the sensitivity of cells to insulin, an alpha glucosidase inhibitors to decrease the absorption of carbohydrates from the intestine, a sitagliptin (dipeptidyl peptidase-4 (DPP-4) inhibitor, such as Januvia® sitagliptin), a biguanide (such as metformin), a SGLT2 inhibitor, a GLP agonist, an incretin mimetic, a dopamine-2 agonist, a bile acid sequestrant, sulfonylurea, or a combination thereof, also are provided. The composition containing the vitamin D3, niacinamide, and lipoic acid as described herein can be administered by any suitable route, including, but not limited to, parenteral, systemic, topical and local, such as by oral, intravenous, intramuscular, mucosal, rectal, and other routes. Formulations suitable for each are provided. The skilled person can establish suitable regimens and doses and select routes of administration.

In practicing the uses and methods herein, the composition containing the blend if vitamin D3, niacinamide, and lipoic acid as described herein can be administered to a subject, including a subject having a pre-diabetic or diabetic condition. One or more steps can be performed prior to, simultaneously with or after administration of the composition to the subject including, but not limited to, diagnosing the subject with a condition appropriate for administering the composition, and determining the blood glucose and HbA1C levels of the subject.

The typical dosage regimen can include administering a single dose of the composition once a day with food. For example, the dosage can be administered with breakfast, lunch or dinner. The dosage can be administered before, during, or after the largest meal of the day. Preferably, the dosage is taken after the largest meal of the day. The dosage regime can include daily dosing over a period of several days, several weeks, or can be administered for several years. Administration can occur daily for at least 2 weeks, or at least 1 month, or at least 2 months, or at least 3 months. Administration can occur for extended periods of time, such as for 1 year, or 2 years, or 5 years, or 10 years or more.

As discussed above, the uses and methods provided herein also can include administering one or more further therapeutic compounds to the subject in addition to administering the composition containing the vitamin D3, niacinamide, and lipoic acid as described herein to the subject. The further therapeutic agent, can be one or a combination of insulin, a meglitinide to increase the insulin output by the pancreas, a thiazolidinedione to increase the sensitivity of cells to insulin, an alpha glucosidase inhibitors to decrease the absorption of carbohydrates from the intestine, a sitagliptin (dipeptidyl peptidase-4 (DPP-4) inhibitor, such as Januvia), a biguanide (such as metformin), a SGLT2 inhibitor, a GLP agonist, an incretin mimetic, a dopamine-2 agonist, a bile acid sequestrant, sulfonylurea, or a combination thereof. The further therapeutic agents can be provided together or separately, such as prior to, contemporaneous with, or after administration of the composition containing the vitamin D3, niacinamide, and lipoic acid. The composition containing the vitamin D3, niacinamide, and lipoic acid can act independently, or in conjunction with the further therapeutic agent, to achieve therapeutic effects.

The methods provided herein can further include one or more steps of monitoring the subject. Any of a variety of monitoring steps can be included in the methods provided herein, including, but not limited to, monitoring blood glucose levels, and measuring blood HbA1C levels, of a subject.

The purpose of the monitoring can be simply for assessing the health state of the subject or the progress of therapeutic treatment of the subject, or can be for determining whether or not further administration of the same or a different further therapeutic agent warranted, or for determining when or whether or not to administer a further therapeutic agent to the subject where the compound can act to increase the efficacy of the therapeutic method, or to determine whether administering the composition containing the vitamin D3, niacinamide, and lipoic acid alone is sufficient to achieve or maintain the targeted therapeutic result, such as reduced blood glucose levels and/or blood HbA1C levels.

Provided is a method of lowering blood glucose level in an animal that is diabetic, non-diabetic, or pre-diabetic. The method includes administering to the animal a therapeutically effective amount of the nutraceutical composition provided herein. The method results in a reduction of fasting blood glucose level in the animal. The fasting blood glucose level can be reduced in the animal to less than 100 mg/dL or less after 25 weeks, or after 20 weeks, or after 15 weeks, or after 10 weeks of administration of the composition provided herein.

Also provided is a method of lowering a plasma level of HbA1C in an animal that is diabetic, non-diabetic, or pre-diabetic. The method includes administering to the animal a therapeutically effective amount of the nutraceutical composition provided herein. The plasma level of HbA1C in the animal can be reduced by at least 0.25 percentage point after 10 weeks of administration of the composition provided herein. The plasma level of HbA1C in the animal can be reduced by at least 0.5 percentage point, or at least 0.75 percentage point, or at least 1 percentage point after 10 weeks of administration of the composition provided herein. The plasma level of HbA1C in the animal can be reduced by more than 1 percentage point after 15 weeks of administration of the composition provided herein.

In the methods provided herein, the animal can be a primate, such as a human, a gorilla or a monkey; a rodent, such as mice and rats; fowl, such as chickens or turkeys; a ruminant, such as goats, cows, deer, and sheep; a porcine; a feline; or a canine. The animal can be a non-human animal. The animal can be a mammal.

Also provided are methods for delaying onset of or preventing diabetic retinopathy in a subject, or delaying onset of or preventing diabetic neuropathy in a subject, or delaying onset of or preventing diabetic nephropathy in a subject. The subject can be diabetic, or non-diabetic, or pre-diabetic. The methods include administering to the subject a therapeutically effective amount of the nutraceutical composition provided herein, wherein the HbA1C level is reduced by at least 0.45 percentage point after 12 weeks of treatment. The plasma level of HbA1C in the subject can be reduced by at least 0.5 percentage point, or at least 0.75 percentage point, or at least 1 percentage point after 15 weeks of administration of the composition provided herein. The plasma level of HbA1C in the animal can be reduced by more than 1 percentage point after 15 weeks of administration of the composition provided herein. Because the HbA1C and blood glucose levels can be controlled in the subject, the risk of complications such as cardiovascular disease, retinopathy, nephropathy, and neuropathy can be reduced or their onset delayed.

In the methods provided herein, the nutraceutical composition can be administered to a subject by oral, intravenous, intramuscular, intraperitoneal, subcutaneous, rectal, mucosal, or transdermal administration.

In the methods provided herein, the nutraceutical composition containing the combination of vitamin D3, niacinamide and lipoic acid provided herein, can be administered in a dosage form selected from among a tablet, a soft gel, a capsule, a pill, a caplet, a dragée, a lozenge, a troche, an effervescent tablet, an emulsion, a chewing gum, a chewable gel or gummy, a wafer, an orally disintegrating tablet, a sublingual tablet, a buccal tablet, a buccal patch, an orodispersible film, granules, a powder, a spray, a solution, a syrup, and a suppository. The dosage form of the composition administered to the subject can include, in addition to the ternary blend of active ingredients, one or a combination of a binder, carrier material, a color agent, a disintegrant, an effervescence agent, an excipient, a filler, a flavor, a lubricant, a surfactant, a suspending agent, and a sweetener. In some embodiments, the pharmaceutical composition can be administered in a dosage form that is a capsule. The capsule can include the ternary blend of active ingredients and one or a combination of a binder, a carrier material, an excipient, or a filler within a gelatin or vegetable capsule. The capsule can include pellets, beads, granules or minitablets containing the ternary blend of active ingredients of the composition provided herein inside the capsule. In some embodiments, the methods include administering the nutraceutical composition in the form of a single tablet formed of a blend, an aggregate, pellets, beads, or granules that are compressed to form the tablet.

In a subject that is diabetic, the methods can include administering the nutraceutical composition containing the combination of vitamin D3, niacinamide and lipoic acid provided herein in conjunction with another therapeutic agent. The another therapeutic agent can be selected from among insulin, a meglitinide, a thiazolidinedione, an alpha glucosidase inhibitor, a dipeptidyl peptidase-4 (DPP-4) inhibitor, a biguanide, a SGLT2 inhibitor, a GLP agonist, an incretin mimetic, a dopamine-2 agonist, a bile acid sequestrant, and sulfonylurea. The nutraceutical composition provided herein enhances glucose uptake from blood into muscles, which helps to lower blood sugar.

### Working of the Composition of the Invention

The combination of vitamin D3, niacinamide and lipoic acid provided herein improves insulin sensitivity and enhances glucose uptake from blood into muscles, which helps to lower blood sugar. This TRIPLE ACTION of the combination provided herein helps pre-diabetics and type 2 diabetics maintain healthy blood glucose levels when used together with exercise, diet and/or medication.

The present combination is different from other supplements on the market for glucose control in three ways:
1) Unique first time product of two approved supplements which when combined together work synergistically to lower glucose levels;
2) Triple action of our product is based on science and laboratory studies; and
3) Established Safety of Vitamin D3, Niacinamide and lipoic acid which are deemed Generally Regarded As safe (GRAS) by US FDA for daily use. These doses are greater than the dose indicated in the combination product provided herein. The typical dose of these vitamins/lipoic acid sold as over the counter supplements typically are around 5000 IU per day for Vitamin D3, 500 mg per day for niacinamide, and 500 mg per day for lipoic acid.

The composition provided herein that is a synergistic combination vitamin D3, niacinamide, and lipoic acid, is meant for anyone with a family history of diabetes, or with a sedentary life style, or who is overweight, or who is diagnosed with pre-diabetes or type 2 diabetic, or any combination of these factors.

A typical dose of the composition provided herein that is a synergistic combination vitamin D3, niacinamide, and lipoic acid is a unit dosage containing precise combination (500 IU of Vitamin D3: 12-120 mg of Niacinamide: 50-100 mg of lipoic acid) of Vitamin D3, Niacinamide and lipoic acid that can be taken once a day, preferably after a meal. The dosage can be taken after the largest meal of the day. The composition provided herein can be used with any other medicines a subject may be taking for treating diabetes.

### Advantages of the Invention

1. The fixed dose combination (FDC) of vitamin D3, niacinamide and lipoic acid which provide synergistic interaction to provide glucose control
2. The FDC promotes muscle glucose uptake, muscle energy production, and increased kinetics of uptake
3. Combination of the FDC with other diabetes medicines
4. Formulations of the FDC including, tablets, oral suspensions, capsules, chewing gum

### EXAMPLES

The composition provided herein will be described in detail by way of the following examples. However, these examples are for illustrative purposes only, and are not intended to limit the scope of the present disclosure.

### Example 1. Capsule Preparation

This example provides a preferred, but not limited, dosage form of a composition of the invention. Vegan capsules (size 1) were produced by preparing an embodiment of the nutraceutical composition provided herein. Each of the following components:

| Component | Weight (g) |
|---|---|
| Vitamin D3 (100 IU/mg) | 90 |
| Niacinamide | 145 |
| Alpha-lipoic acid | 600 |
| Excipients (combination of dicalcium | 1465 |
| phosphate anhydrous, colloidal silicon | |
| dioxide, and magnesium sterate) | |

was sifted through a screen having a preselected mesh size to produce components that have the desired particle size. The components then were blended together for a period of time from 10 to 30 minutes to produce a uniform blend. The blend was transferred to a capsule filling machine and the machine was operated using standard operating procedures to fill size 1 vegan capsules. Each capsule was filled with 230 mg of the blend of components.

These capsules can be administered in a regimen effective to prevent or treat pre-diabetes or diabetes, or for lowering blood glucose levels and/or HbA1C in a subject. The capsule can be taken once a day, preferably before, during, or after the largest meal of the day.

### Example 2. Glucose Uptake in an In Vitro Model

The effect of each of vitamin D3, niacinamide and lipoic acid separately, and the fixed dose combination of vitamin D3, niacinamide and lipoic acid composition provided herein, were evaluated for the ability of each to have an effect on glucose uptake in an in vitro model. A goal was to improve insulin's ability to increase the uptake of glucose by L6 muscle cells.

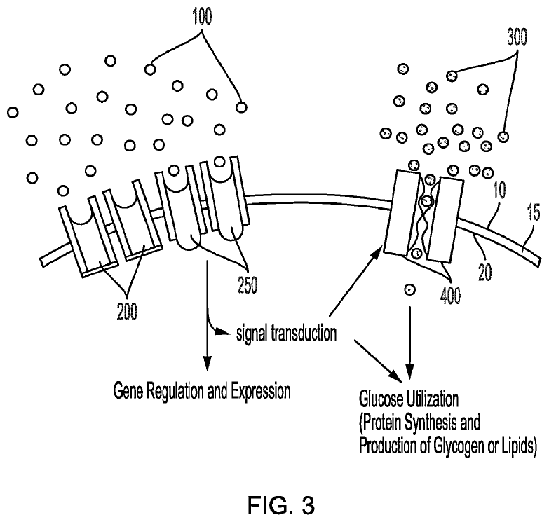

Two specific molecular events in glucose uptake were targeted: 1) increasing GLUT4 levels in the cells to enhance glucose transport into the cells by insulin, and 2) utilizing the glucose for energy generation in the mitochondria where, using the TCA cycle, glucose is metabolized to generate ATP by using niacinamide (a precursor of nicotinamide adenine dinucleotide, NAD, a co-enzyme in TCA cycle). Replenishment of NAD can further boost glucose uptake and utilization. GLUT4 glucose transport is depicted in FIG. 3. In FIG. 3, 10 is the outside of the cell and 20 is the inside of the cell, and 15 is the cell membrane. Insulin receptors 200 are inactive until they combine with insulin 100, resulting in activated insulin receptors 250 that promote signal transduction, resulting in gene expression and growth regulation. One of the signals resulting from activated insulin receptors 250 stimulates redistribution of GLUT4 glucose transporter 400 from an intracellular distribution within intracellular vesicles to translocation to the plasma membrane, where is can catalyze the transport of glucose 300 across the cell membrane 15 through an ATP-independent, facilitative diffusion mechanism. (Huang et al., Cell Metabolism 5:237-252 (2007)).

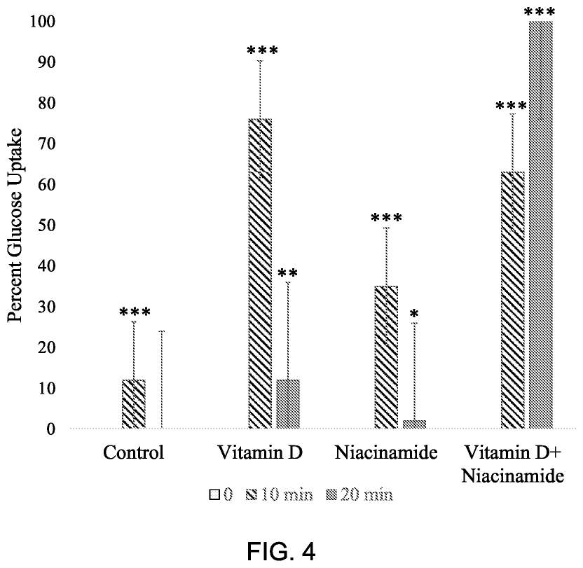

Vitamin D3 was utilized to increase GLUT4 levels since this vitamin has been shown before to increase GLUT4 levels in muscle, and niacinamide was used to increase cellular NAD levels (El-Fakhri et al., Horm Res Paediact 81:363-378 (2014); Manna et al., J Biol Chem 287 (50): 42324-42332 (2012); Garten, Endocrinologist, Issue 135 (2020); and Fan et al., J Diabetes Investig. 11 (6): 1403-1419 (2020)). Glucose uptake studies using L6 cells were performed. L6 cells (ATCC CLR-1458; Rockville, MD) were maintained in Dulbecco's modified Eagle's medium (DMEM) supplemented with 10% Fetal Bovine Serum (FBS) and 100 μg/mL penicillin and streptomycin, at 37° C. in an atmosphere of 5% CO₂. 2-(N-[7-nitrobenz-2-oxa-1, 3-diazol-4-yl]amino)-2-deoxyglucose (2-NBDG) (Molecular Probes-Invitrogen, CA, USA) was used to assess glucose uptake in the L6 cells. The cells were kept in glucose free medium for half an hour before insulin stimulation. The cells were then stimulated with 100 nM insulin for 10 and 20 min and incubated with 10 μM of 2-NBDG for 15 min. The reaction was stopped by washing with cold PBS three times and the cells were lysed using 0.1% Triton X. The lysate was then used to read florescence at 535 nM. The data is reported in FIG. 4 as percent of control cells (no treatment, incubation with media).

FIG. 4 shows the percent increase in glucose uptake in the presence of insulin. Experiment conducted n=5. Student's t-tests were performed to compare between the present glucose uptake shown by control, vitamin D, niacinamide, and their combination at different time points.

As shown in FIG. 4, in the presence of insulin in control cells (not treated with vitamin D3 or niacinamide), there was only modest uptake of glucose at 10 minutes and none at 20 minutes after insulin pulse treatment.

When vitamin D3 alone was added to the cells before insulin pulse, glucose uptake after insulin pulse increased by about 6-fold more than control cells at 10 minutes but less so at 20 minutes. This suggest that vitamin D3 alone can increase glucose uptake.

The effect of niacinamide treatment alone also was tested. When niacinamide alone was added to the cells before insulin pulse, glucose uptake after insulin pulse increased by about 3 fold over control at 10 minutes but only little at 20 min, suggesting that this vitamin can also increase glucose uptake on its own.

The combination of vitamin D3 and niacinamide then was tested to determine whether there was a synergistic effect, because they work through different mechanisms. As can be seen in FIG. 4, the combination resulted in a very dramatic increase in glucose uptake, especially at 20 minutes, where the increase was 9 fold higher and more than either of the agents alone. These data suggest a synergistic effect between vitamin D and niacinamide in glucose uptake by muscle cells. The other interesting thing was that the combination sustains the glucose increase peaks 20 minutes unlike when either agent is added alone.

Because the TCA cycle and glucose metabolism in the mitochondria generate oxidative stress, lipoic acid, an antioxidant (Golbidi et al., Front Pharmacol, Vol. 2, Article 69 (2011), 15 pages) which is known to enter the mitochondria, was tested to determine if it could further enhance glucose uptake. The following combinations were tested for improvements in muscle cell glucose uptake stimulated by insulin: Vitamin D3 plus Niacinamide plus or minus Lipoic acid alone.

As shown in FIG. 5, relative to control cells and as expected from above studies, the combination of Vitamin D3 and niacinamide (V=N) showed significant increase in glucose uptake relative to control cells. In contrast Lipoic acid alone did not increase glucose uptake alone but the combination of Vitamin D3, Niacinamide and Lipoic acid (LA+V+N) increased glucose uptake even moderately better than Vitamin D3 and Niacinamide combination. Interestingly, Vitamin E a powerful antioxidant but which does not enter the mitochondria, had no effect. These data support the idea of improvement in glucose metabolism by suppressing mitochondrial oxidative stress. The data shown in FIG. 5 are statistically significant for the V+N and LA+V+N from control p>0.05.

### Example 3. Glucose Uptake in an In Vivo Model

Oral glucose tolerance test (OGTT) is often used to test the effect of an intervention on disposal of orally given glucose as a measure of the body's ability to clear glucose from the circulation. The lower the glucose levels after a glucose meal, the more effective is the physiological system including insulin, muscle, liver and adipose etc in glucose uptake by the organs thus lowering blood glucose.

In order to test the effect of the composition provided herein in vivo, male Wistar rats were used in an OGTT study. The animals (control group treated with vehicle (n=6 animals) and rats administered the composition provided herein (vitamin mix treated group, (n=6 animals) were treated with the mixture dissolved in ethanol/water as vehicle. The animals were dosed once a day orally for two weeks and then given an oral bolus of glucose and blood was collected at various time points and glucose levels were measured.

The composition provided herein also was tested in an in vitro model. Oral Glucose tolerance test in male wistaria rats was performed as is known in the art (Seshadri et al., Scientific Reports 9, id. 8825 (2019), 9 pages). The studies were performed by Liveon Biolabs PVT Ltd under IAEC approval. The protocol used briefly was as follows: animals were acclimatized and were randomly distributed into 6 groups, each group comprised of 6 rats. The rats in group G1 serves as normal control group and administered with Vehicle alone at the dose volume of 10 mL/kg whereas the rats in G4 group were administered with Vitamin mix respectively at the doses of 200 mg/kg body weight for single administration. respectively at the dose of half capsule a day for two weeks. All the Rats were observed for clinical signs, morbidity, mortality, body weight and blood glucose were measured during the study period.

### Test System Details

Animal Species: Rat (Rattus norvegicus)
Strain: Wistar Rats
Age at treatment: 7-8 weeks
Total number of animals: 12
Sex: Male
Number of animals per group: 6
Number of groups: 2
Bodyweight range: Around 250 grams

### Animal Room Preparation

Prior to housing the animals, the experimental room was decontaminated by fumigation and microbial load was checked by settle plate method. The experimental room floor was mopped with disinfectant daily once.

### Husbandry Conditions

Animals were housed in an environment-controlled room at temperature of 20.1-22.5° C. and relative humidity of 52-68%. The photoperiod was 12 hours fluorescent light and 12 hours darkness. Adequate fresh air supply of 12-15 air changes/hour and sound level of <80 dB was maintained in the experimental room. The relative humidity, Maximum and minimum temperature in the experimental room were recorded once daily. The copies of results is included in the study file.

### Housing

Two rats per cage were housed in standard polycarbonate cage (size: approximately Length 43×Breadth 29×Height 18 cm) with stainless steel top grill having facilities for pelleted food and drinking water. During the study period, rats were housed in a single experimental room. Polycarbonate rat tunnels were provided to the animals as an environmental enrichment objects in the cages that either provide shelter or exercising opportunities to minimize animal stress and promote overall well-being. These enrichment objects were replaced at least once a week.

### Test Procedure

Wistar Rats were randomly divided into 2 groups (vehicle only and test item). For two weeks vehicle (ethanol+water) or test item was administered to the respective groups. After 30 min of last dose, glucose was administered in the dose 2 gm/kg. 0 min (before glucose administration), after glucose administration 30 min, 60 min, 90 min, 120 min and 180 min was measured glucose level by using Accu Chek glucometer. OGTT study was conducted on overnight fasting animals. The dose formulation preparation were prepared freshly before administration and were used within 2 hours of formulation preparation.

### 2 Week Repeated Dose OGTT Study:

Vitamin mixture consisting of RDA levels of Vitamin D3, Niacinamide and lipoic acid (500 IU vitamin D3, 12 mg niacinamide, and 50 mg alpha-lipoic acid) was dissolved in 2 ml of ethanol (50%) and 1 ml of water, and half of the volume was used to dose the animals orally for two weeks.

### Diet and Water

AF-1000M R&M diets manufactured by Krishna Valley Agrotech LLP was provided ad libitum to rats. Deep bore-well water subjected to reverse osmosis and UV sterilized, was provided ad libitum to rats in polycarbonate bottles with stainless rubber corked steel sipper tubes. The food and water provided to the Rats were tested for contaminants.

### Acclimatization

The animals were acclimatized to experimental room conditions for a period of 6 days prior to initiation of treatment. Body weights were recorded at the initiation and end of acclimatization period. Animals were observed for mortality and morbidity once daily during acclimatization period. Veterinary examination was performed before selecting the animals and only healthy and active animals were used in the study.

### Randomization and Grouping

Grouping of animals was performed on the fifth day of acclimatization by body weight randomization and stratification method and allocated into two groups G1 and G4 groups. The body weight variation within the groups of animals did not exceed ±20% of the mean body weight. Body weights of the animals were analyzed statistically for mean body weight to rule out the statistically significant difference between groups.

### In Vivo Study Compliance

The study was performed as per the mutually agreed Study Plan and the Standard Operating Procedures (SOPs) of the Test Facility. The use of animals for this study had been approved by Liveon Biolabs Private Limited Institutional Animal Ethics Committee (IAEC).

### Animal Welfare and Veterinary Care

Liveon Biolabs Private Limited is an AAALAC International accredited facility and registered with CPCSEA, Department of Animal Husbandry and Dairying (DAHD), Ministry of Fisheries, Animal Husbandry and Dairying (MoFAH&D), Government of India. Also, Liveon Biolabs Private Limited ensures that animal experiments are performed in accordance with the recommendation of the regulatory guidelines for laboratory animal facility published in the gazette of India, 2021.

During the conduct of study none of the animals were injured, and no moribund animals were observed.

TABLE 1
Summary of blood glucose levels in animals after Oral Glucose Tolerance Test

| | | | | | | | | |
|---|---|---|---|---|---|---|---|---|
| G4 | Male | Mean | 116.83 | 177.00 | 171.50 | 165.33 | 148.17 | 115.00 |
| (Normal | | SD | 12.43 | 5.44 | 4.97 | 6.50 | 11.70 | 7.27 |
| control) | | | | | | | | |
| Minutes | | | 0 minutes | 30 | 60 | 90 | 120 | 180 |
| after oral | | | 0 | 30 | 60 | 90 | 120 | 180 |
| glucose | | | | | | | | |
| load | | | | | | | | |
| G6 | | Mean | 117.83 | 161.33** | 155.33** | 147.00*** | 132.50** | 112.83 |
| (Vitamin | | SD | 4.17 | 7.03 | 11.93 | 8.44 | 10.78 | 3.06 |
| Mix) | | | | | | | | |

The values are expressed in Mean ± SD: (n = 6)
*P < 0.01,
**P < 0.001,
***P < 0.0001 vs. control group

TABLE 2
Summary of Area Under the Curve (AUC) values for blood glucose

| | | | Time in Minutes | | | | | |
|---|---|---|---|---|---|---|---|---|
| Groups | Sex | | 0 | 30 | 60 | 90 | 120 | 180 |
| G4 | Male | Mean | 0.00 | 9.79 | 11.62 | 11.23 | 10.45 | 4.39 |
| (Normal | | SD | 0.00 | 0.50 | 0.31 | 0.35 | 0.54 | 0.21 |
| control) | | | | | | | | |
| Minutes | | | 0 | 30 | 60 | 90 | 120 | 180 |
| after oral | | | 0 | 30 | 60 | 90 | 120 | 180 |
| glucose | | | | | | | | |
| load | | | | | | | | |
| G6 | | Mean | 0.00 | 9.31* | 10.56*** | 10.08*** | 9.32*** | 4.09 |
| (Vitamin | | SD | 0.00 | 0.21 | 0.43 | 0.67 | 0.62 | 0.17 |
| Mix) | | | | | | | | |

The values are expressed in Mean ± SD: (n = 6)
*P < 0.01,
**P < 0.001,
***P < 0.0001 as compared to induction control (G4) group

As shown in the data, after oral glucose administration, load at every time point examined, there was statistically significant decrease in the vitamin treated group in glucose levels relative to the vehicle treated control group. These data suggest that the composition provided herein has improved the glucose disposal system from circulation and resulted in glucose lowering.

The calculated Area Under the Curve (ACU) of glucose levels from 0-180 minutes time point also showed a statistically significant decrease suggesting that the composition provided herein effect was sustained and successfully blunted the rise in blood glucose after oral glucose load.

### Example 4. Clinical Trial

To further evaluate the efficacy of the composition provided herein, an ongoing 3-month clinical study in 30 diabetic and prediabetic participants was conducted. The following study protocol was used.

| | |
|---|---|
| Study Phase and Design | Observational/Prospective |
| | Open label pilot study |
| Study Medication and Dosing | An embodiment of the nutraceutical composition |
| | provided herein for oral dosing, formulated as a |
| | powder in a capsule - 500 IU vitamin D, 50 mg |
| | alpha-lipoic acid, and 12 mg niacinamide per capsule |
| | (FDC). |
| | One capsule consumed orally daily along with the |
| | main meal of the day |
| Comparator/Baseline | Subjects' fasting blood glucose levels (over a period |
| | of 2 weeks) prior to use of FDC. |
| Objectives | Primary Endpoint: |
| | 1) Reduction in fasting blood glucose by 10% |
| | following one month of treatment with FDC relative |
| | to baseline. |
| | Secondary Endpoints: |
| | 1) Reduction in fasting blood glucose by 20% |
| | following two months of treatment with FDC relative |
| | to baseline. |
| | 2) Maintenance of reduced fasting blood glucose |
| | levels relative to baseline (by 20%) over an additional |
| | 4 weeks following two months of treatment with FDC |
| | relative to baseline, while on continued daily use of |
| | FDC. |
| | Tertiary Endpoints: |
| | 1) Reduction in currently used medication in diabetic |
| | population. |
| Study Design | No samples will be collected by the sponsor. |
| | Sponsor will collect their diabetes history |
| | information, as well as case history of other |
| | morbidities such as high blood pressure, |
| | kidney or liver disease, recent and chronic |
| | infections etc. |
| | Study participants will test fasting blood |
| | glucose weekly using “at home use test kits” |
| | or other glucose monitoring devices and |
| | record in the diary. |
| | Subjects will record daily events such as |
| | medications, supplements, diet, and any |
| | adverse events. |
| | Subjects will consume one capsule of FDC |
| | orally daily along with the main meal of the |
| | day. |
| | Subjects are expected to keep their normal |
| | routine of diet, medication, exercise. |
| Selection Criteria | Inclusion criteria: |
| (inclusion/exclusion) | This is an all-comer study (for prediabetics |
| | and diabetics). |
| | Age 18+ |
| | Exclusion criteria: |
| | Type 1 diabetics |
| | Pregnant women |
| | Chronic liver or kidney disease |
| | Chronic HBV, HCV, HIV infection |
| | Recent (less than a month) SARS CoV-2 |
| | infection |
| | Recent steroid use (less than a month) |
| | Previous discomfort or adverse events |
| | following use of multivitamins, Vitamin D3, |
| | Vitamin B3 (Niacin) or Niacinamide. |
| Data Analysis | For treatment differences chi-square or t tests will |
| | be used depending on the sample size for all of the |
| | following analyses. |
| | 1. Magnitude changes relative to baseline will be |
| | assessed |
| | 2. For continuous variables, area under the under the |
| | curve analyses will be used. |
| | Primary, Secondary and Tertiary endpoints will |
| | include all of the above analyses. |
| | Missing data will be imputed using a median of all |
| | evaluable assessments at each timepoint. |
| Safety: | The formulation is based on nutraceutical guidance |
| | and the components are generally recognized as Safe |
| | (GRAS) |
| Efficacy/Benefit: | Reduction in fasting blood glucose relative to |
| | baseline, as specified in the endpoints is determined |
| | to be beneficial and the product is considered to be |
| | efficacious. |

We collated data from limited observational studies in patients as observational, i.e. studies where an inference is drawn from a sample patient population is not under the control of the researcher. An embodiment of the nutraceutical composition provided herein was used in patients who were diabetic and were being treated by various anti-diabetic drugs. Diabetic patients were typically being treated with metformin and/or Jardiance® (empagliflozin), semaglutide, or insulin, and the nutraceutical composition provided herein was given in addition to the pharmaceutical treatment. The patient data was collected under an informed consent. Shown here is the data from 8 patients as was made available.

Table 3 shows a dashboard view and Table 4 shows a summary of the patient data reviewed.

TABLE 3
Changes in fasting plasma glucose levels after nutraceutical treatment
Dashboard

| | | | | Post | | |
|---|---|---|---|---|---|---|
| Patient # | Gender | Age | Baseline | treatment | Change | % Change |
| 1 | | 47 | 215 | 137 | 78 | −36% |
| 2 | | 72 | 245 | 149 | 96 | −39% |
| 3 | | 69 | 202 | 169 | 33 | −16% |
| 4 | | — | 177 | 152 | 26 | −14% |
| 5 | | — | 202 | 176 | 26 | −13% |
| 6 | M | 68 | 120 | 101 | 19 | −15% |
| 7 | | — | 119 | 117 | 2 | −2% |
| 8 | | — | 217 | 100 | 117 | −54% |
| Mean | | | | | | −24% |
| Median | | | | | | −16% |
| UL | | | | | | −54% |
| LL | | | | | | −2% |

TABLE 4
One way ANOVA analysis of data
Summary of Data

| | Treatments | | |
|---|---|---|---|
| | | Post- | |
| | Baseline | Treatment | |
| | (Treatment 1) | (Treatment 2) | Total |
| N | 8 | 8 | 16 |
| ΣX | 1497 | 1101 | 2598 |
| Mean | 187.125 | 137.625 | 162.375 |
| ΣX² | 294837 | 157501 | 452338 |
| Std. Dev. | 45.8427 | 29.2181 | 45.0834 |

Result Details

| Source | SS | df | MS | |
|---|---|---|---|---|
| Between-treatments | 9801 | 1 | 9801 | F = 6.63294 |
| Within-treatments | 20686.75 | 14 | 1477.625 | |
| Total | 30487.75 | 15 | | |

The f-ratio value is 6.63294.
The p-value is .022003.
The result is significant at p < .05.

As can be seen from the above observational study data from the available 8 patients, there are some conclusions that can be drawn. The data show that there is an average drop of fasting plasma glucose in most patients, and the decrease on an average is 24% from initiation of treatment with the nutraceutical of the invention, and is statistically significant with p-value <0.022. The decrease is seen on top of other anti-diabetic drugs that were used in addition to the nutraceutical composition provided herein. The decrease in fasting plasma glucose is sustained over a few weeks period and in no case was there a rebound back to before initiation of administering the nutraceutical composition provided herein, suggesting a lasting decrease in fasting blood glucose. The interim analysis of fasting blood sugar in the 8 subjects showed an average reduction in fasting blood sugar in humans that is consistent with the OGTT (Oral Glucose Tolerance Test) preclinical study results in rats. Data for fasting blood glucose and HbA1C profile in one of the subjects is provided in Table 5.

TABLE 5
Changes in fasting blood glucose and HbA1C is a subject administered
FDC (the nutraceutical composition provided herein).

| Fasting Blood | | | HBA1C (%) | | | |
|---|---|---|---|---|---|---|
| Glucose (FBS, mg/dL) | | | Glyco- | HBA1C | | |
| | Blood | | hemoglobin/ | (Relative | With or | Data |
| | glucose, | FBS (Relative | Total | to Pre- | Without | Collection |
| Week | mg/dL | to Pre-FDC) | hemoglobin | FDC) | FDC | Date |
| −5 | 120 | 100.00 | | 100.00 | | 1-May |
| −4 | 124 | | 6.80 | | | 8-May |
| −3 | 121 | | | | | 15-May |
| −2 | 117 | | | | | 22-May |
| −1 | 118 | | | | | 29-May |
| 0 | 116 | 96.67 | | | +FDC | 5-Jun |
| 1 | 110 | 91.67 | | | +FDC | 12-Jun |
| 2 | 113 | 94.17 | 6.50 | 95.59 | +FDC | 19-Jun |
| 3 | 104 | 86.67 | | | +FDC | 26-Jun |
| 4 | 119 | 99.17 | | | +FDC | 3-Jul |
| 5 | 109 | 90.83 | | | +FDC | 10-Jul |
| 6 | 106 | 88.33 | | | +FDC | 17-Jul |
| 7 | 102 | 85.00 | | | +FDC | 24-Jul |
| 8 | 92 | 76.67 | 6.20* | 91.18 | +FDC | 31-Jul |
| 9 | 98 | 81.67 | | | +FDC | 7-Aug |
| 10 | 95 | 79.17 | | | +FDC | 14-Aug |
| 11 | 97 | 80.83 | | | +FDC | 21-Aug |
| 12 | 98 | 81.67 | 5.80 | 85.29 | +FDC | 28-Aug |
| 14 | 97 | 80.83 | | | +FDC | 12-Sep |
| 16 | 92 | 76.67 | | | +FDC | 25-Sep |
| 18 | 97 | 80.83 | 5.80* | 85.29 | +FDC | 13-Oct |
| 20 | 98 | 81.67 | | | +FDC | 30-Oct |
| 25 | 98 | 81.67 | | | +FDC | 4-Dec |

*Fructosamine-based calculation

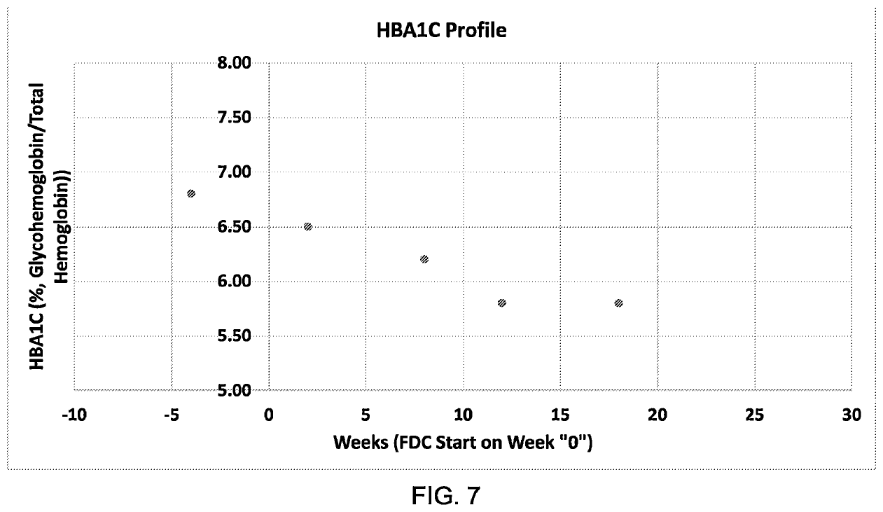

The results for the fasting blood glucose values are shown graphically in FIG. 6, and the results for the HbA1C profile are shown graphically in FIG. 7. As can be seen in FIG. 6, the fasting blood glucose level is reduced by about 10% after 5 weeks of administration of the nutraceutical composition provided herein, and blood glucose levels below 100 mg/dL were achieved and maintained after about 10 weeks of administering the nutraceutical product provided herein. As can be seen in FIG. 7, HbA1C is reduced by more than 0.25 percentage point after 8 weeks of treatment with the nutraceutical composition, and is reduced by 0.7 percentage point after 12 weeks of treatment with the nutraceutical composition provided herein.

Two of the subjects (patients 7 and 8) were being treated with 2 grams metformin per day. The results of fasting blood glucose after 1 month of treatment with the nutraceutical composition provided herein in addition to the metformin is shown in Table 6.

TABLE 6
Results in subjects also being treated with metformin

| Before FDC Treatment | After FDC Treatment |
|---|---|
| 119 mg/ml | 117 |
| 217 | 100 |

As can be seen from the data, the reduction in fasting blood glucose is achieved in subjects treated with another anti-diabetic drug. One of the subjects exhibited a significant improvement in fasting blood glucose after 1 month of treatment with the nutraceutical composition provided herein.

1. A nutraceutical composition for treating or preventing the complications of pre-diabetes or diabetes in a subject, comprising:
a ternary blend of 500 IU of vitamin D3, 12-120 mg of niacinamide, and 50-100 mg of lipoic acid as active ingredients.

2-40. (canceled)

41. The nutraceutical composition of claim 1, wherein the only active ingredients are the vitamin D3, niacinamide, and lipoic acid.

42. The nutraceutical composition of claim 41, wherein:
the ratio of the vitamin D3 to the niacinamide is from about 50:1 to 50:12.

43. The nutraceutical composition of any one of claim 42, wherein:
the ratio of the vitamin D3 to the lipoic acid is from about 10:1 to 5:1.

44. The nutraceutical composition of any one of claim 43, wherein:
the ratio of the niacinamide to the lipoic acid is from about 0.12:1 to 2.4:1.

45. The nutraceutical composition of claim 44, wherein:
the lipoic acid is R-alpha-lipoic acid; or
the lipoic acid is S-alpha-lipoic acid; or
the lipoic acid is a blend of R-alpha-lipoic acid and S-alpha-lipoic acid.

46. The nutraceutical composition of claim 1, wherein the composition is in a form selected from among a tablet, a soft gel, a capsule, a pill, a caplet, a dragée, a lozenge, a troche, an effervescent tablet, an emulsion, a chewing gum, a chewable gel product, a wafer, an orally disintegrating tablet, a sublingual tablet, a buccal tablet, a buccal patch, an orodispersible film, granules, a powder, a spray, a solution, a syrup, and a suppository.

47. The nutraceutical composition of claim 1, further comprising one or a combination of a binder, carrier material, a color agent, a disintegrant, an effervescence agent, an excipient, a filler, a flavor, a lubricant, a surfactant, a suspending agent, and a sweetener.

48. The nutraceutical composition of claim 46, further comprising one or a combination of a binder, carrier material, a color agent, a disintegrant, an effervescence agent, an excipient, a filler, a flavor, a lubricant, a surfactant, a suspending agent, and a sweetener.

49. A method of treating, delaying the onset of or preventing pre-diabetes, diabetes mellitus, diabetic retinopathy, diabetic neuropathy or diabetic nephropathy in a mammal in need thereof, comprising administering to the mammal a therapeutically effective amount of the nutraceutical composition of claim 1, wherein after 12 weeks of administration an average decrease of a fasting blood glucose level is 20% or more based on fasting plasma glucose level prior to administration, or wherein the HbA1C level is reduced by at least 0.45 percentage point after 12 weeks of treatment.

50. The method of claim 49, wherein the subject is non-insulin dependent.

51. The method of claim 49, wherein the nutraceutical composition is administered by oral, intravenous, intramuscular, intraperitoneal, subcutaneous, rectal, mucosal, or transdermal administration.

52. The method of claim 49, wherein the nutraceutical composition is administered in a dosage form selected from among a tablet, a soft gel, a capsule, a pill, a caplet, a dragée, a lozenge, a troche, an effervescent tablet, an emulsion, a chewing gum, a chewable gel or gummy, a wafer, an orally disintegrating tablet, a sublingual tablet, a buccal tablet, a buccal patch, an orodispersible film, granules, a powder, a spray, a solution, a syrup, and a suppository.

53. The method of claim 49, wherein the nutraceutical composition is administered once a day.

54. The method of claim 53, wherein the nutraceutical composition is administered before, during, or after consuming the largest meal of the day.
